(19) United States
(12) Patent Application Publication
(10) Pub. No.: US 2004/0106962 A1
(43) Pub. Date: Jun. 3, 2004

(54) Implantable stimulation device and method for adjusting AV/PV delay according to patient's posture

(76) Inventors: Junyu Mai, Valencia, CA (US); Euljoon Park, Stevenson Ranch, CA (US); Kerry A. Bradley, Glendale, CA (US)

Correspondence Address: PACESETTER, INC., 15900 Valley View Court, Sylmar, CA, 91392-9221, US

(21) Appl. No.: 10/722,971

(22) Filed: Nov. 25, 2003

Related U.S. Application Data
(63) Continuation of application No. 09/627,528, filed on Jul. 28, 2000, now abandoned.

(51) Int. Cl. A61N 1/365

(52) U.S. Cl. 607/19

(57) ABSTRACT

An implantable cardiac stimulation device and method applies pacing stimulation pulses to a ventricle of a heart with an AV delay adjusted according to the patient's posture. The device includes a pulse generator that delivers pacing stimulation pulses to the ventricle upon the expiration of an AV delay. A posture detector senses posture of the patient and a processor adjusts the AV delay responsive to the sensed posture of the patient.

### CROSS-REFERENCE TO RELATED APPLICATIONS

This application is a continuation of copending U.S. patent application Ser. No. 09/627,528, filed Jul. 28, 2000.

### FIELD OF THE INVENTION

The present invention is generally directed to an implantable cardiac stimulation device. The present invention is more particularly directed to such a device having an AV delay, which is adjusted depending upon the posture of the patient.

### BACKGROUND OF THE INVENTION

Dual-chamber pacing is well known in the art. A major benefit of dual-chamber pacing is the capability of providing a pacing regime that closely approximates the natural synchrony between atrial and ventricular contractions. One important parameter to this natural synchrony is the AV delay or interval.

In a healthy heart, atrial contractions or activations (P waves) are followed by ventricular contractions or activations (R waves). The time between the P wave and R wave permits ventricular filling by the P wave. Generally, the time interval between P waves and R waves decreases with increased heart rate when the individual is more active to satisfy the increased hemodynamic demand.

In a pacemaker, the AV delay or interval mimics the time interval between P waves and R waves of a healthy heart. The AV delay, in a demand pacemaker, is the time between an atrial contraction, either natural or paced, and the delivery of a ventricular pacing stimulus in the absence of a natural or intrinsic R wave within the AV delay.

To simulate the varying PR intervals of a healthy heart, pacemakers are able to adjust the AV delay. In the past, this has been accomplished in rate responsive pacemakers where the pacing rate is increased when the patient is more active and decreased when the patient is less active. Also, as the pacing rate is increased, the AV delay is decreased, and when the pacing rate is decreased, the AV delay is increased.

The degree of activity is generally sensed by an activity sensor such as an accelerometer or a vibration sensor. Hence, in the prior art, the AV delay has been varied responsive to the sensed activity of the patient.

Other arrangements for varying the AV delay are also known. For example, one arrangement contemplates setting the AV delay responsive to the QT interval of the heart when the patient is at rest. The AV delay is then varied from that setting responsive to patient activity.

In another known arrangement, the AV delay is varied as a function of mitral regurgitation sounds produced by the heart when the patient is at rest. This has been advanced as being particularly useful for treating Hypertrouphic Obstructive Cardiomyopathy (HOCM).

None of the foregoing arrangement takes the patient's posture into account when adjusting the AV delay. More specifically, optimal AV delay is different with a patient's posture, even with the same heart rate. This results in an AV delay which may not be appropriate when the patient has a posture that is different from what it is when AV delay is adjusted. For example, if the AV delay is adjusted when the patient is sleeping in bed, it may be too long for the patient when sitting or standing.

### SUMMARY OF THE INVENTION

The present invention therefore provides an implantable cardiac stimulation device and method wherein the AV delay of the device is adjusted by the posture of the patient. A posture detector senses the posture of the patient between an upright position and a lying down position. In accordance with one embodiment, the AV delay is selected from a first preset AV delay corresponding to the patient being in an upright position and a second AV delay corresponding to the patient being in a lying down position.

In accordance with a further embodiment, an optimal AV delay is set while the patient is in a lying down position. Thereafter, the AV delay is increased or decreased depending upon the posture of the patient.

In accordance with a still further embodiment of the present invention, the AV delay is varied between a first AV delay when the patient is in an upright position and a second AV delay when the patient is in a lying down position.

Once the AV delay is adjusted according to the patient's posture, the AV delay may thereafter be varied or adjusted according to the activity of the patient.

### BRIEF DESCRIPTION OF THE DRAWINGS

The above and other aspects, features, and advantages of the present invention will be more apparent from the following more particular description thereof presented in conjunction with the following drawings and wherein:

FIG. 1 shows a simplified functional block diagram of a combined implantable cardioverter/defibrillator (ICD) and pacemaker, which represents one type of implantable stimulation device with which the present invention may be used;

FIG. 2 a functional block diagram of an implantable dual-chamber pacemaker, which represents another type of implantable medical device with which the invention may be used;

FIG. 3 is a flowchart that illustrates the method used to perform AV delay adjustments in accordance with one embodiment of the present invention; and

FIG. 4 is a flowchart that illustrates another method used to perform AV delay adjustments in accordance with another embodiment of the present invention.

### DETAILED DESCRIPTION OF THE PREFERRED EMBODIMENTS

The following description is of the best mode presently contemplated for carrying out the invention. This description is not to be taken in a limiting sense, but is made merely for the purpose of describing the general principles of the invention. The scope of the invention should be determined with reference to the claims.

As indicated above, the present invention may be used with various types of implantable stimulation devices, including an implantable pacemaker configured to treat bradycardia and/or tachycardia, or an implantable cardioverter-defibrillator (ICD) combined with an implantable pacemaker.

To better understand the invention, it will first be helpful to have an understanding of the basic functions performed by the implantable stimulation device with which the invention is used, e.g., an ICD device and/or a dual-chamber pacemaker. While a dual-chamber device has been chosen, this is for teaching purposes only. It is recognized that the present invention could be implemented into any device which at least senses in an atrium, paces in a ventricle, and has an AV delay. All such devices are considered to be within the spirit of the invention.

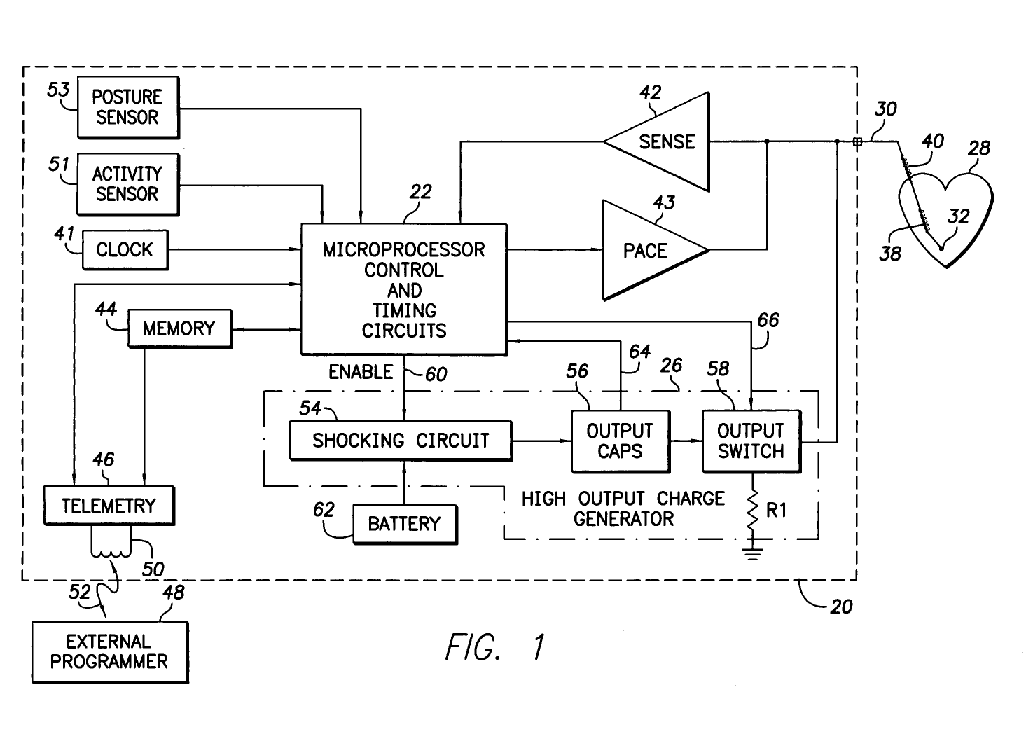

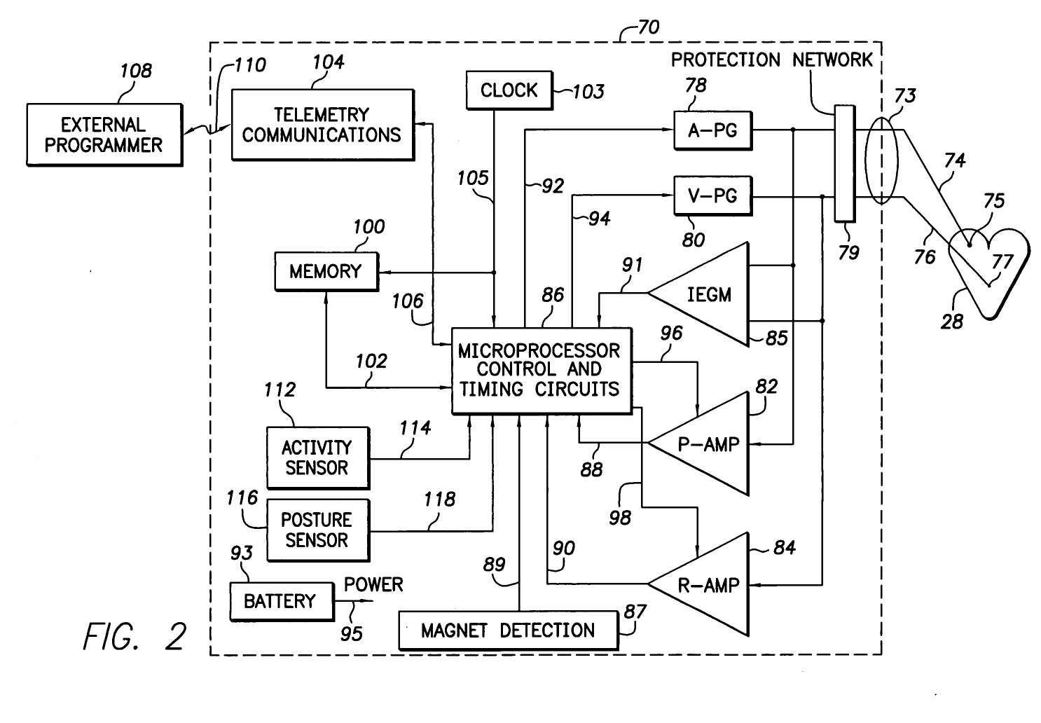

In FIG. 1, there is shown a simplified functional block diagram of an ICD device 20, and in FIG. 2, there is shown a simplified functional block diagram of a dual-chamber pacemaker 70. It should also be noted that the ICD 20 includes a pacing circuit 43 to combine the functionality of an ICD and a pacemaker within the same stimulation device.

It is the primary function of an ICD device to sense the occurrence of an arrhythmia, and to automatically apply an appropriate electrical shock therapy to the heart aimed at terminating the arrhythmia. To this end, the ICD device 20, as shown in the functional block diagram of FIG. 1, includes a control and timing circuit 22, such as a microprocessor, state-machine or other such control circuitry, that controls the ICD and pacemaker functions of the device 20.

With respect to the ICD function, a high output charge generator 26 is controlled by the circuit 22. The high output charge generator 26 generates electrical stimulation pulses of moderate or high energy (corresponding the cardioversion or defibrillation pulses, respectively), e.g., electrical pulses having energies of from 1 to 10 joules (moderate) or 11 to 40 joules (high), as controlled by the control/timing circuit 22.

Such moderate or high energy pulses are applied to the patient's heart through at least one lead 30 having at least two defibrillation electrodes, such as coil electrodes 38 and 40. The lead 30 preferably also includes at least one electrode for pacing and sensing functions, such as electrode 32. Typically, the lead 30 is transvenously inserted into the heart so as to place the coil electrodes 38 and 40 in the apex of the heart and in the superior vena cava, respectively. While only one lead is shown in FIG. 1, it is to be understood that additional defibrillation leads and electrodes may be used as desired or needed in order to efficiently and effectively apply the shock treatment generated by the high voltage generator 26 to the patient's heart 28.

The ICD 20 also includes a sense amplifier 42. It is the function of the sense amplifier 42 to sense the electrical activity of the heart 28, as is known in the art, such as R waves which occur upon the depolarization, and hence contraction, of ventricular tissue; and P waves which occur upon the depolarization, and hence contraction, of atrial tissue. Thus, by sensing R waves and/or P waves through the sense amplifier 42, the control/timing circuit 22 is able to make a determination as to the rate and regularity of the patient's heart beat and whether a pacing stimulation pulse should be delivered to the heart. Such information, in turn, also allows the control/timing circuit 22 to determine whether the heart 28 of a patient is experiencing a tachyarrhythmia, and to apply appropriate anti-tachyarrhythmia stimulation therapy.

The control/timing circuit 22 further has a memory circuit 44 coupled thereto wherein the operating parameters and instructions used by the control/timing circuit 22 are stored. Such operating parameters define, for example, the amplitude of each shock energy pulse to be delivered to the patient's heart 28 within each tier of therapy, as well as the duration of these shock pulses. The operating instructions define the method steps performed by circuit 22 to implement the ICD and pacemaker functions. The memory 44 may take many forms, and may be subdivided into as many different memory blocks or sections (addresses) as needed to store desired data and control information.

Advantageously, the operating parameters of the implantable device 20 may be non-invasively programmed into the memory 44 through a telemetry circuit 46, in telecommunicative contact with an external programmer 48 by way of a suitable coupling coil 50. The coupling coil 50 may serve as an antenna for establishing a radio frequency (RF) communication link 52 with the external programmer 48; or the coil 50 may serve as a means for inductively coupling data to and from the telemetry circuit 46 from and to the external programmer 48, as is known in the art. See, e.g., U.S. Pat. No. 4,809,697 (Causey, III et al.) and U.S. Pat. No. 4,944,299 (Silvian), incorporated herein by reference. Further, such telemetry circuit 46 advantageously allows status information relating to the operation of the ICD 20, as contained in the control/timing circuit 22 or memory 44, to be sent to the external programmer 48 through the established link 52.

The control/timing circuit 22 includes appropriate processing and logic circuits for analyzing the output of the sense amplifier 42 and determining if such signals indicate the presence of an arrhythmia. Typically, the control/timing circuit 22 is based on a microprocessor, or similar processing circuit, which includes the ability to process or monitor input signals (data) in a prescribed manner, e.g., as controlled by program code stored in a designated are or block of the memory 44. The details of the design and operation of the control/timing circuit 22 are not critical to the present invention. Rather, any suitable control/timing circuit 22 may be used that carries out the functions described herein. The use, design, and operation of microprocessor-based control circuits to perform timing and data analysis functions is known in the art.

The pacing pulse generator 43 may be of the type as described subsequently with respect to FIG. 2. It may provide for the delivery of pacing stimulation pulses to both the atria and ventricles. The control circuit 22, in analyzing the activity sensed by sensing circuit 42, provides for demand pacing. Only when there is an absence of natural R wave or P wave within an escape interval is a pacing pulse delivered.

One such escape interval is the AV delay or interval, also referred to as the AV/PV delay interval. The AV delay is the interval which begins with an atrial contraction, either natural or paced, and ends when a ventricular pacing pulse is to be delivered absent the occurrence of a natural R wave. As will be seen hereinafter, the device 20 adjusts the AV/PV delay according to posture of the patient.

The device 20 further includes an activity sensor 51 that is connected to the control circuit 22. While the sensor 51 is illustrated in FIG. 1 as being included within the device 20, it is to be understood that the sensor may also be external to the device yet still be implanted within or carried by the patient. A common type of activity sensor is an accelerometer or piezoelectric crystal, that is mounted to the case of the device. Other types of sensors are also known, such as sensors that sense the oxygen content of blood, respiration rate, pH of blood, body motion, and the like. The type of sensor used is not critical to the present invention. Any sensor or combination of sensors capable of sensing a physiological or physical parameter relatable to the rate at which the heart should be beating (i.e., relatable to the metabolic need of the patient), and/or relatable to whether a tachyarrhythmia is likely to soon occur, can be used. Such sensors are commonly used with “rate-responsive” pacemakers in order to adjust the rate and the AV delay of the pacemaker in a manner that tracks activity of the patient.

In accordance with the present invention, the device 20 further includes a posture sensor 53. The posture sensor detects the posture of the patient between a fully upright position and a lying down position. To that end, the sensor 53 may include accelerometers which detect acceleration in three mutually transverse directions. The raw signals from the sensor 53 are provided to the control circuit 22 which may generate to different control signals. A first control signal may be a logical “1” if the patient is in an upright position and a logical “0” if the patient is in a lying down position. A second control signal may be a multiple-bit binary fractional factor between 0 and 1 representing the posture of the patient. For example, the fractional factor may vary from 0, representing the patient in a lying down position, to 1, representing the patient in a fully upright position. One such posture sensor is fully described in copending U.S. application Ser. No. 09/457,451, filed Dec. 8, 1999, entitled “AN AC/DC MULTI AXIS ACCELEROMETER FOR DETERMINING PATIENT ACTIVITY AND BODY POSITION,” now U.S. Pat. No. 6,466,821, which patent is owned by the assignee of the present invention and incorporated herein it its entirety by reference.

Various methods for adjusting the AV delay, in accordance with the present invention, will be described in detail subsequently. In general, however, the AV delay will be longer when the patient is in a lying down position and shorter when the patient is in a fully upright position. Once the AV delay is adjusted according to the patient's posture, it may thereafter be varied from the posture adjusted AV delay based upon patient activity or pacing rate.

In FIG. 2, a simplified block diagram of the circuitry needed for a dual-chamber pacemaker 70 is illustrated. The pacemaker 70 is coupled to a heart 28 by way of leads 74 and 76, the lead 74 having an electrode 75 that is in contact with one of the atria of the heart, and the lead 76 having an electrode 77 that is in contact with one of the ventricles of the heart. The leads 74 and 76 are electrically and physically connected to the pacemaker 70 through a connector 73 that forms an integral part of the housing wherein the circuits of the pacemaker are housed.

The connector 73 is electrically connected to a protection network 79, which network 79 electrically protects the circuits within the pacemaker 70 from excessive shocks or voltages that could appear on the electrodes 75 and/or 77 in the event such electrodes were to come in contact with a high voltage signal, e.g., from a defibrillation shock.

The leads 74 and 76 carry stimulating pulses to the electrodes 75 and 77 from an atrial pulse generator (A-PG) 78 and a ventricular pulse generator (V-PG) 80, respectively. Further, electrical signals from the atria are carried from the electrode 75, through the lead 74, to the input terminal of an atrial channel sense amplifier (P-AMP) 82; and electrical signals from the ventricles are carried from the electrode 77, through the lead 76, to the input terminal of a ventricular channel sense amplifier (R-AMP) 84. Similarly, electrical signals from both the atria and ventricles are applied to the inputs of an IEGM (intracardiac electrogram) amplifier 85. The amplifier 85 is typically configured to detect an evoked response from the heart 28 in response to an applied stimulus, thereby aiding in the detection of “capture.” (Capture occurs when an electrical stimulus applied to the heart is of sufficient energy to depolarize the cardiac tissue, thereby causing the heart muscle to contract, or in other words, causing the heart to beat. Capture does not occur when an electrical stimulus applied to the heart is of insufficient energy to depolarize the cardiac tissue.)

The dual-chamber pacemaker 70 is controlled by a control system 86 that typically includes a microprocessor programmed to carry out control and timing functions. The control system 86 receives the output signals from the atrial (P-AMP) amplifier 82 over signal line 88. Similarly, the control system 86 receives the output signals from the ventricular (R-AMP) amplifier 84 over signal line 90, and the output signals from the IEGM amplifier 85 over signal line 91. These output signals are generated each time that a P wave or an R wave or an evoked response is sensed within the heart 28. The control system 86 also generates trigger signals that are sent to the atrial pulse generator (A-PG) 78 and the ventricular pulse generator (V-PG) 80 over signal lines 92 and 94, respectively. These trigger signals are generated each time that a stimulation pulse is to be generated by the respective pulse generator 78 or 80. The atrial trigger signal is referred to simply as the “A-trigger,” and the ventricular trigger signal is referred to as the “V-trigger.”

During the time that either an A-pulse or V-pulse is being delivered to the heart, the corresponding amplifier, P-AMP 82 and/or R-AMP 84, is typically disabled by way of a blanking signal presented to these amplifiers from the control system over signal lines 96 and 98, respectively. This blanking action prevents the amplifiers 82 and 84 from becoming saturated from the relatively large stimulation pulses that are present at their input terminals during this time. This blanking action also helps prevent residual electrical signals present in the muscle tissue as a result of the pacemaker stimulation from being interpreted as P waves or R waves.

As shown in FIG. 2, the pacemaker 70 further includes a memory circuit 100 that is coupled to the control system 86 over a suitable data/address bus 102. This memory circuit 100 allows certain control parameters, used by the control system 86 in controlling the operation of the pacemaker, to be programmably stored and modified, as required, in order to customize the pacemaker's operation to suit the needs of a particular patient. Further, data sensed during the operation of the pacemaker may be stored in the memory 100 for later retrieval and analysis.

As with the memory 44 of the ICD device 20 shown in FIG. 1, the memory 100 of the pacemaker 70 (FIG. 2) may take many forms. It may be subdivided into as many different memory blocks or sections (addresses) as needed in order to allow desired data and control information to be stored.

A clock circuit 103 directs an appropriate clock signal(s) to the control system 86, as well as to any other needed circuits throughout the pacemaker 70 (e.g., to the memory 100) by way of clock bus 105.

A telemetry/communications circuit 104 is further included in the pacemaker 70. This telemetry circuit 104 is connected to the control system 86 by way of a suitable command/data bus 106. In turn, the telemetry circuit 104, which is included within the implantable pacemaker 70, may be selectively coupled to an external programming device 108 by means of an appropriate communication link 110, which communication link 110 may be any suitable electromagnetic link, such as an RF (radio frequency) channel, a magnetic link, and inductive link, an optical link, and the like. Advantageously, through the external programmer 108 and the communication link 110, desired commands may be sent to the control system 86. Similarly, through this communication link 110 with the programmer 108, data commands (either held within the control system 86, as in a data latch, or stored within the memory 100) may be remotely received from the programmer 108. Similarly, data initially sensed through the leads 74 or 76, and processed by the microprocessor control circuits 86, or other data measured within or by the pacemaker 70, may be stored and uploaded to the programmer 108. In this manner, non-invasive communications can be established with the implanted pacemaker 70 from a remote non-implanted, location.

The pacemaker 70 additionally includes a battery 93. The battery 93 provides operating power to all of the circuits of the pacemaker 70 via a POWER signal line 95.

It is noted that the pacemaker 70 in FIG. 2 is referred to as a dual-chamber pacemaker because it interfaces with both the atria and the ventricles of the heart. Those portions of the pacemaker 70 that interface with the atria, e.g., the lead 74, the P wave sense amplifier 82, the A-PG 78, and corresponding portions of the control system 86, are commonly referred to as the “atrial channel.” Similarly, those portions of the pacemaker 70 that interface with the ventricles, e.g., the lead 76, the R wave sense amplifier 84, the V-pulse generator 80, and corresponding portions of the control system 86, are commonly referred to as the “ventricular channel.”

The pacemaker 70 further includes an activity sensor 112 that is connected to the control system 86 of the pacemaker 70 over a suitable connection line 114. The sensor 112 may be of the type as previously described with respect to sensor 51 of FIG. 1.

The pacemaker 70 further includes magnet detection circuitry 87, coupled to the control system 86 over signal line 89. It is the purpose of the magnet detection circuitry 87 to detect when a magnet is placed over the pacemaker, which magnet may be used by a physician or other medical personnel to perform various reset functions of the pacemaker 70, and/or to signal the control system 86 that an external programmer 108 is in place to receive data from, or send data to, the pacemaker memory 100 or control system 86 through the telemetry communications circuits 104.

As with the ICD device 20 of FIG. 1, the telemetry or communications circuit 104 may be of conventional design, such as is described in U.S. Pat. No. 4,944,299, or as is otherwise known in the art. Similarly, the external programmer 108 may be of any suitable design known in the art, such as is described in U.S. Pat. No. 4,809,697. Likewise, the memory circuit 100, and the circuits utilized in the atrial and ventricular channels may all be of common design as is known in the pacing art. The present invention is not concerned with the details of the circuitry utilized for each of these pacing elements. Rather, it is concerned with the manner in which all of these pacing elements cooperate with each other in order to provide a particular pacing mode of operation. Such cooperation is controlled by the control system 86.

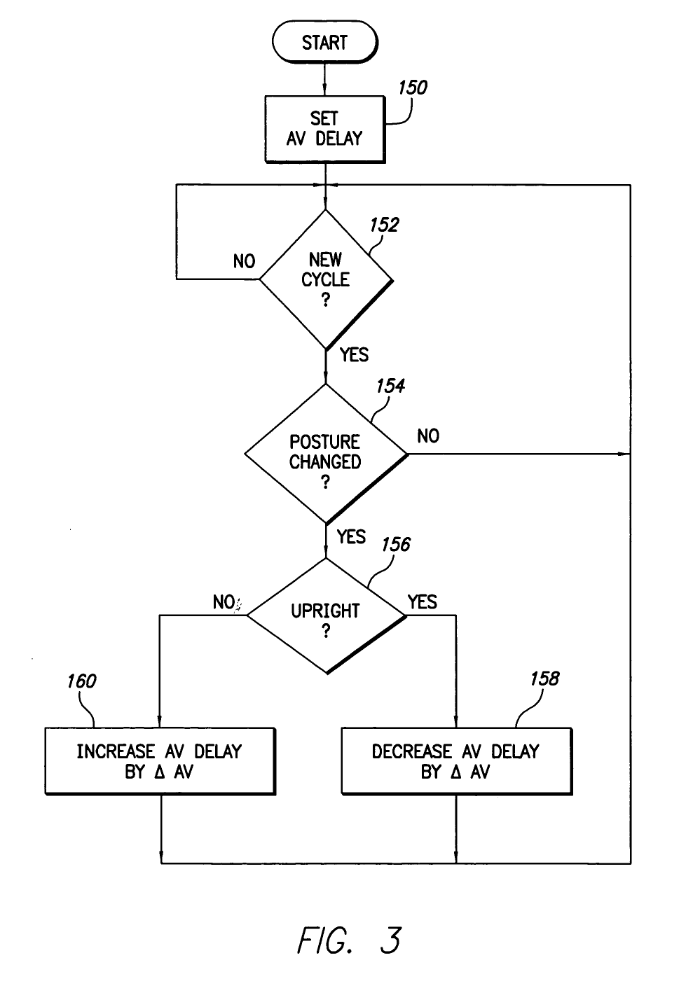

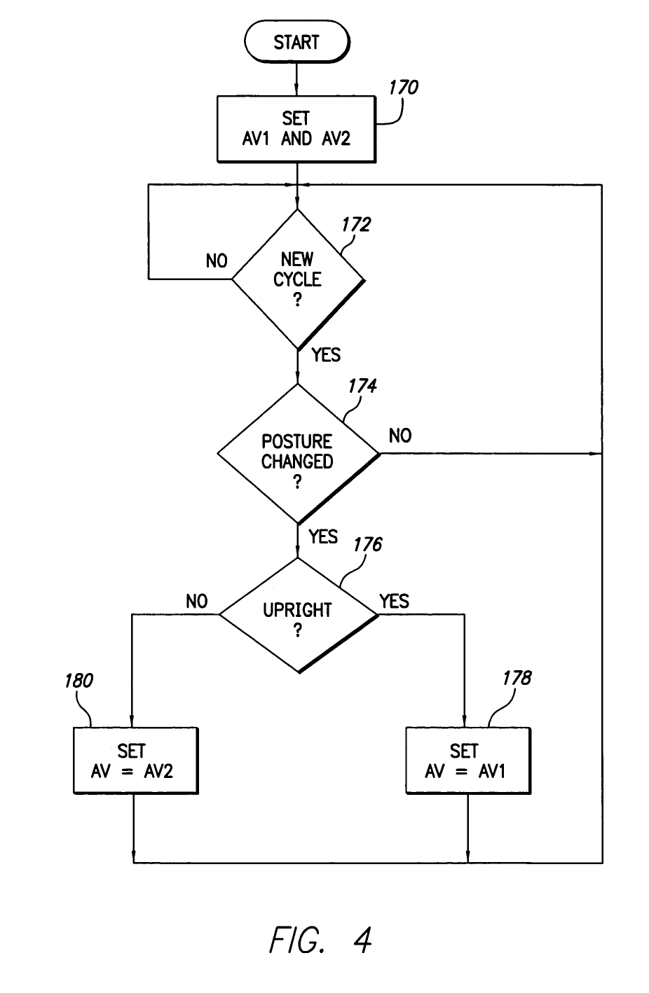

The control system 86 may be realized using a variety of different techniques and/or circuits. The preferred type of control system 86 is a microprocessor-based control system. It is noted, however, that the control system 86 could also be realized using a state machine. Indeed, any type of control circuit or system could be employed for the control system 86. The present invention is likewise not concerned with the details of the control systems 22 and 86. Rather, it is concerned with the end result achieved by the control system. That is, so long as the control system 86 controls the operation of the pacemaker (or other medical device) so that the desired functions are achieved as set forth herein, e.g., by following the steps described below in the flow charts of FIGS. 3 and 4, it matters little what type of control system is used. Those of skill in the implantable medical device art, given the teachings presented herein, should thus be able to fashion numerous different types of control systems or circuits that achieve the desired device control.

Representative of the types of control systems that may be used with the invention is the microprocessor-based control system described in U.S. Pat. No. 4,940,052, entitled “Microprocessor Controlled Rate-Responsive Pacemaker Having Automatic Rate Response Threshold Adjustment.” Reference is also made to U.S. Pat. Nos. 4,712,555 and 4,944,298, wherein a state-machine type of operation for a pacemaker is described; and U.S. Pat. No. 4,788,980, wherein the various timing intervals used within the pacemaker and their inter-relationship are more thoroughly described. The '052, '555, '298 and '980 patents are incorporated herein by reference.

The pacemaker 70 still further includes, in accordance with the present invention, a posture sensor 116 which is coupled to the control circuit 86 over a suitable connection 118. The posture sensor 116 may be of the type as previously described with respect to the posture sensor 53 of FIG. 1. The flowcharts of FIGS. 3 and 4 illustrate methods which may be employed, in accordance with the present invention, for adjusting the AV delay of the pacing circuit 43 of FIG. 1 or the pacemaker 70 of FIG. 2 in response to the posture sensors 53 and 116 respectively.

Referring now to FIG. 3, the method there illustrated may be implemented by either the control circuit 22 of FIG. 1 or the control circuit 86 of FIG. 2. The method initiates with an activity block 150 wherein an optimal AV delay is set, for example at implantation, by the physician while the patient is in a lying down position. The AV delay is preferably selected by the physician using the external programmer and transferred to the device using the telemetry circuits previously described.

Once The AV delay is set in accordance with activity block 150, the method proceeds to a decision block 152 wherein it is determined if the heart is in a new cardiac cycle. If the heart is not, the control circuit will wait until a new cycle begins.

Once a new cycle begins, the process proceeds to another decision block 154 wherein it is determined if the posture of the patient has changed. If the posture of the patient has not changed, the method returns to step 152 because no adjustment in the AV delay due to posture is required. A change in the posture of the patient may be discerned by the control circuit by monitoring the logical 1 or logical 0 state presently derived from the raw posture signal provided by the posture sensor 53 with the state developed during the previous cardiac cycle. Hence, if the state has changed from a logical 0 to a logical 1 or a logical 1 to a logical 0, the posture will have been considered to have changed.

If the posture of the patient has changed since the last cardiac cycle, the process then proceeds to a decision block 156 wherein it is determined if the patient is now upright. If the control circuit developed a logical 1 based upon the current posture data provided by the posture sensor 53 or 116, the patient will be considered to have changed from a lying down position to an upright position. This causes the method to advance to an activity step 158 wherein the AV delay is decreased. The decrease in the AV delay may be by a fixed amount previously determined by the physician.

If in decision block 156 it is determined that the patient is not upright and hence has moved from an upright position to a lying down position, the method advances to activity block 160 wherein the AV delay is increased. The increase in AV delay is the same amount as the decrease in activity block 158.

Following the adjustment of the AV delay, the method then returns to decision block 152 to repeat the foregoing process for the next cardiac cycle.

Referring now to FIG. 4, it illustrates another method in which the AV delay may be adjusted responsive to the posture of the patient. The method of FIG. 4 initiates at step 170 wherein the physician sets a first AV delay (AV 1) for the patient in an upright position and a second AV delay (AV 2) for the patient in a lying down position. Next, the method advances to decision block 172 wherein it is determined if the heart has begun a new cardiac cycle. Again, if the heart has not begun a new cardiac cycle, the method waits until a new cardiac cycle has begun.

When a new cardiac cycle begins as determined in accordance with decision block 172, the method advances to decision block 174 wherein it is determined if the posture has changed. Decision block 174 may be carried out in the same manner previously described with respect to decision block 154 of FIG. 3. If the posture of the patient has not changed, the method returns to step 172 to wait for a new cardiac cycle to begin since no adjustment in AV delay is necessary. However, if the posture of the patient has changed as determined in accordance with decision block 174, the method advances to decision block 176 to determine if the patient is now upright. If the patient is now upright, the method advances to activity block 178 wherein the AV delay is set to the first AV delay (AV 1) selected by the physician for the patient in the upright position. However, if the patient is now in a lying down position, the method transitions from decision block 176 to activity block 180 wherein the AV delay is set to the second AV delay (AV 2) previously selected by the physician for the patient being in a lying down posture. Following either of activity blocks 178 or 180, the method then returns to decision block 172 to await for the beginning of a new cardiac cycle.

While the methods illustrated in FIGS. 3 and 4 contemplate varying the AV delay by a fixed amount or setting the AV delay to fixed values depending upon the posture of the patient, in accordance with a still further aspect of the present invention, the AV delay may be gradually changed between an AV delay for the patient in a lying down posture to the AV delay when the patient is in a fully upright posture. In accordance with this aspect of the present invention, the physician may select two optimal AV delays, for example, a first AV delay (AV 1) for the patient being in a fully upright posture and a second AV delay (AV 2) for the patient being in a fully lying down posture. The AV delay may then be changed as a function of the current fractional factor determined by the control circuit as previously described. For example, the AV delay may be gradually varied between AV 1 and AV 2 in accordance with the relationship below:

AV(N)=AV2+P*(AV1−AV2)

where P is a current fractional factor between 0 and 1.

Once the AV delay is adjusted for the posture of the patient, it may thereafter be varied responsive to the activity of the patient or the pacing rate. As a result, by using posture information to adjust the AV delay, optimum hemodynamic performance of the implanted cardiac stimulation device may be achieved.

While the invention has been described by means of specific embodiments and applications thereof, it is understood that numerous modifications and variations could be made thereto by those skilled in the art without departing from the spirit and scope of the invention. It is therefore to be understood that within the scope of the claims, the invention may be practiced otherwise than as specifically described herein.

What is claimed is:

1. An implantable cardiac stimulation device for applying pacing stimulation pulses to a ventricle of a patient, the device comprising:
a pulse generator that is operative to generate the pacing stimulation pulses for delivery to the ventricle;
a posture detector that is operative to sense posture of the patient; and
control circuitry that is connected to the pulse generator and to the posture detector and that controls generation of the pacing stimulation pulses, wherein the control circuitry is operative to select an atrio-ventricular delay based solely on the sensed posture of the patient, wherein the control circuitry is operative to decrease the atrio-ventricular delay when the patient goes from a lying down position to an upright position, and to increase the atrio-ventricular delay when the patient goes from an upright position to a lying down position.

2. The device of claim 1, wherein the control circuitry is operative to select a first atrio-ventricular delay when the patient is in a first position and a second atrio-ventricular delay when the patient is in a second position.

3. The device of claim 1, further comprising an activity sensor that senses activity of the patient and wherein the control circuitry is operative to vary a pacing rate based on the sensed activity.

4. An implantable cardiac stimulation device comprising:
pulse generating means for generating pacing stimulation pulses;
posture detecting means for sensing posture of the patient; and
control means for selecting an atrio-ventricular delay based solely on the sensed posture of the patient, wherein the control means comprises means for decreasing the atrio-ventricular delay when the patient goes from a lying down position to an upright position, and for increasing the atrio-ventricular delay when the patient goes from an upright position to a lying down position.

5. The device of claim 4, wherein the control means adjusts the atrio-ventricular delay by selecting a first atrio-ventricular delay when the patient is in a first position and a second atrio-ventricular delay when the patient is in a second position.

6. The device of claim 4, further comprising activity sensing means for sensing activity of the patient and wherein the control means varies a pacing rate based on the activity sensing means.

7. A method of applying pacing stimulation pulses to a ventricle of a patient, the method comprising:
sensing posture of the patient;
adjusting an atrio-ventricular delay based solely on the sensed posture of the patient; and
wherein adjusting the atrio-ventricular delay comprises decreasing the atrio-ventricular delay when the patient goes from a lying down position to an upright position, and increasing the atrio-ventricular delay when the patient goes from an upright position to a lying down position.

8. The method of claim 7, wherein adjusting comprises selecting a first atrio-ventricular delay when the patient is in a first position and a second atrio-ventricular delay when the patient is in a second position.

9. The method of claim 7, further comprising sensing activity of the patient and varying a pacing rate based on the activity sensor.
